(19) United States
(12) Patent Application Publication
(10) Pub. No.: US 2006/0241434 A1
(43) Pub. Date: Oct. 26, 2006

(54) Ultrasonic image construction method and diagnostic ultrasound apparatus

(75) Inventor: Tadashi Shimazaki, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/374,865

(22) Filed: Mar. 14, 2006

(30) Foreign Application Priority Data
Mar. 16, 2005 (JP) 2005-075083

(51) Int. Cl. A61B 8/00

(52) U.S. Cl. 600/437

(57) ABSTRACT

An object of the present invention is to make it possible to observe the whole of a diagnostic region covering a wide range and to improve diagnostic efficiency. Based on a first echo signal produced by scanning with ultrasonic waves a three-dimensional field corresponding to a first position on a subject, a first image of the three-dimensional field corresponding to the first position is constructed in the form of a C-mode image. Thereafter, based on a second echo signal produced by scanning a three-dimensional field corresponding to a second position with an ultrasonic probe that is moved from the first position on the subject to the second position, a second image of the three-dimensional field corresponding to the second position is constructed in the form of a C-mode image. Thereafter, the first and second images are joined so that they will be associated with the first and second positions respectively, whereby a joint image is constructed. The joint image is then displayed on a display surface.

### BACKGROUND OF THE INVENTION

The present invention relates to an ultrasonic image construction method and a diagnostic ultrasound apparatus.

Diagnostic ultrasound apparatuses produce an echo signal by transmitting ultrasonic waves to a subject and receiving the ultrasonic waves reflected from the subject, construct an image of a subject's section on the basis of the produced echo signal, and then display the image of the section on a screen. The diagnostic ultrasound apparatus supports various imaging modes such as an A mode, a B mode, a C mode, a color flow mapping (CFM) mode, and a pulsed-wave Doppler (PWD) mode. The diagnostic ultrasound apparatus can construct and display an image in real time and is therefore proved useful in the field of medicine, or more particularly, in prenatal screening or cardiac screening.

The diagnostic ultrasound apparatus produces an echo signal by performing a scan to transmit ultrasonic waves from an ultrasonic probe to a three-dimensional field in a subject and to receive ultrasonic waves reflected from the three-dimensional field, constructs an image of the three-dimensional field in real time on the basis of the echo signal, and then displays the image on a display surface (refer to, for example, Patent Document 1).

[Patent Document 1] Japanese Unexamined Patent Publication No. 2000-152932

[Patent Document 2] Japanese Unexamined Patent Publication No. 2001-353150

However, since only an image of a field corresponding to a section scanned with an ultrasonic probe is constructed and displayed on a display surface, the whole of a diagnostic region covering a wide range cannot be observed. Therefore, a scan must be performed again in some cases. This hinders diagnostic efficiency from improving. For example, when the diagnostic region is a thoracic region or a vascular region, the whole of the diagnostic region cannot be scanned. The drawback of poor diagnostic efficiency becomes outstanding.

### SUMMARY OF THE INVENTION

Therefore, an object of the present invention is to provide an ultrasonic image construction method and a diagnostic ultrasound apparatus making it possible to observe the whole of a diagnostic region covering a wide range and to improve diagnostic efficiency.

In efforts to accomplish the above object, an ultrasonic image construction method in accordance with the present invention constructs an image of a subject on the basis of an echo signal produced by transmitting ultrasonic waves from an ultrasonic probe to the subject and receiving the ultrasonic waves reflected from the subject using the ultrasonic probe. The ultrasonic image construction method comprises: a first step of constructing a first image of a three-dimensional field corresponding to a first position on the subject on the basis of a first echo signal produced by performing a scan to transmit ultrasonic waves from the ultrasonic probe to the three-dimensional field corresponding to the first position and to receive the ultrasonic waves reflected from the three-dimensional field corresponding to the first position; and a second step of constructing a second image of a three-dimensional field corresponding to a second position on the basis of a second echo signal produced by performing a scan to transmit the ultrasonic waves to the three-dimensional field corresponding to the second position from the ultrasonic probe moved from the first position on the subject to the second position, and to receive the ultrasonic waves reflected from the three-dimensional field corresponding to the second position, and then constructing a joint image by joining the first image constructed at the first step and the second image so that the images will be associated with the first and second positions respectively.

In efforts to accomplish the aforesaid object, a diagnostic ultrasound apparatus in accordance with the present invention constructs an image of a subject on the basis of an echo signal produced by transmitting ultrasonic waves to a subject from an ultrasonic probe and receiving ultrasonic waves reflected from the subject using the ultrasonic probe. The diagnostic ultrasound apparatus comprises: a transceiver that acquires a first echo signal produced by performing a scan to transmit the ultrasonic waves to a three-dimensional field corresponding to a first position on the subject from the ultrasonic probe and to receive the ultrasonic waves reflected from the three-dimensional field corresponding to the first position, and that then acquires a second echo signal produced by performing a scan to transmit the ultrasonic waves to a three-dimensional field corresponding to a second position from the ultrasonic probe moved to the second position adjoining the first position on the subject and to receive the ultrasonic waves reflected from the three-dimensional field corresponding to the second position; and an image construction unit that constructs a first image of the three-dimensional field corresponding to the first position on the basis of the first echo signal, and that constructs a second image of the three-dimensional field corresponding to the second position on the basis of the second echo signal. The image construction unit constructs a joint image by joining the first image and the second image so that the images will be associated with the first and second positions respectively.

According to the present invention, there is provided an ultrasonic image construction method and a diagnostic ultrasound apparatus making it possible to observe the whole of a diagnostic region covering a wide range and to improve diagnostic efficiency.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the overall configuration of a diagnostic ultrasound apparatus 1 in accordance with the first embodiment of the present invention.

FIG. 2 is a flowchart describing actions to be performed in order to display an image of a subject after scanning the subject using the diagnostic ultrasound apparatus 1 in accordance with the first embodiment of the present invention.

FIG. 3 includes perspective views illustrating scans of a subject performed with the ultrasonic probe 11 included in the diagnostic ultrasound apparatus 1 in accordance with the first embodiment of the present invention, FIG. 3a shows a case where a three-dimensional field R1 corresponding to a first position P1 on a subject is scanned, and FIG. 3b shows a case where a three-dimensional field R2 corresponding to the second position P2 on the subject is scanned.

FIG. 4 shows construction of a joint image IK by joining first and second images I1 and I2 according to the first embodiment of the present invention, FIG. 4a is a perspective view showing the relationship of a plane associated with the first and second images I1 and I2 constructed in the form of C-mode images and a subject's blood vessel visualized in the C-mode images, and FIG. 4b shows the joint image IK constructed by joining the first and second images I1 and I2.

FIG. 5 is a perspective view showing the joint image IK constructed by an image construction unit 13 included in the second embodiment of the present invention.

### DETAILED DESCRIPTION OF THE INVENTION

Embodiments of the present invention will be described below.

### FIRST EMBODIMENT

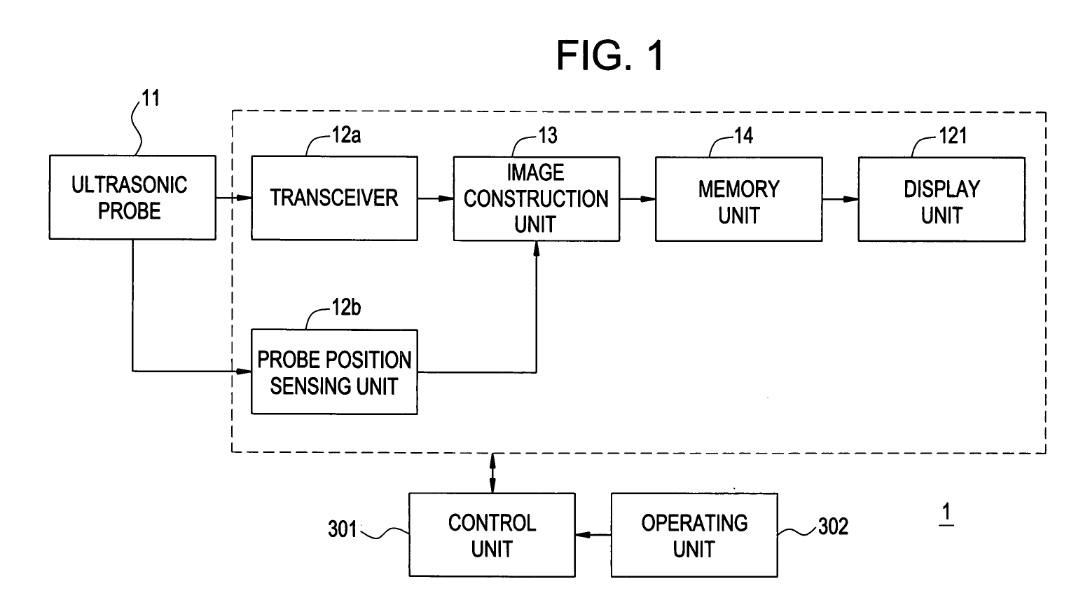

FIG. 1 is a block diagram showing the overall configuration of a diagnostic ultrasound apparatus 1 in accordance with the first embodiment of the present invention.

As shown in FIG. 1, the diagnostic ultrasound apparatus 1 comprises an ultrasonic probe 11, a transceiver 12a, a probe position sensing unit 12b, an image construction unit 13, a memory unit 14, a display unit 121, a control unit 301, and an operating unit 302.

The components of the diagnostic ultrasound apparatus 1 will be described sequentially.

The ultrasonic probe 11 has a contact surface that is abutted on the surface of a subject, transmits ultrasonic waves to the subject through the contact surface, and receives the ultrasonic waves reflected from the subject through the contact surface. The ultrasonic probe 11 is, for example, a two-dimensional array probe and has a plurality of ultrasonic transducers set in array on the surface thereof that is abutted on the surface of the subject. The ultrasonic transducers included in the ultrasonic probe 11 are made of, for example, a lead zilconate titanate (PZT) ceramic. The ultrasonic transducers convert an electric signal into ultrasonic waves, transmit the ultrasonic waves to a subject, receive ultrasonic waves reflected from the subject, and convert the received ultrasonic waves into an electric signal. Specifically, the ultrasonic probe 11 transmits ultrasonic waves produced by the ultrasonic transducers 11a to a subject's intracorporeal region according to a driving signal that is sent from the transceiver 12a in response to a command issued from the control unit 301. Ultrasonic waves reflected from the intracorporeal region to which the ultrasonic waves have been transmitted are received in order to produce an echo signal, and the echo signal is transmitted to the transceiver 12a. Moreover, a magnetic sensor (not shown) included in the probe position sensing unit 12b that will be described later is disposed in the ultrasonic probe 11.

The transceiver 12a includes a transmission/reception circuit that transmits or receives ultrasonic waves. The transceiver 12a is connected to the ultrasonic probe 11. The transceiver 12a causes the ultrasonic transducers included in the ultrasonic probe 11 to transmit ultrasonic waves to a subject in response to a command issued from the control unit 301. The transceiver 12a causes the ultrasonic transducers included in the ultrasonic probe 11 to receive ultrasonic waves reflected from the subject so as to produce an echo signal. For example, the transceiver 12a implements a scan according to an electronic convex scanning method. The transceiver 12a acquires a produced echo signal and transfers the echo signal to the image construction unit 13. Specifically, the transceiver 12a sequentially changes the selected ones of the ultrasonic transducers included in the ultrasonic probe 11 and drives the selected ultrasonic transducers so that the selected ultrasonic transducers will receive ultrasonic waves so as to produce an echo signal, and thus causes the ultrasonic probe 11 to move an ultrasonic beam during a scan. The echo signal is subjected to processing such as amplification, delay, and addition, and then transferred to the image construction unit 13.

Actions will be detailed later. In the present embodiment, the transceiver 12a acquires a first echo signal produced by performing a scan to transmit ultrasonic waves from the ultrasonic probe 11 to a three-dimensional field corresponding to a first position on a subject and to receive ultrasonic waves reflected from the three-dimensional field corresponding to the first position. Thereafter, the transceiver 12a acquires a second echo signal produced by performing a scan to transmit ultrasonic waves to a three-dimensional field corresponding to a second position from the ultrasonic probe 11 that is manually moved to the second position adjoining the first position on the subject and to receive ultrasonic waves reflected from the three-dimensional field corresponding to the second position. Thus, the transceiver 12a sequentially acquires echo signals produced by performing scans to transmit ultrasonic waves to three-dimensional fields corresponding to different positions on a subject from the ultrasonic probe 11 that is sequentially moved to the different positions, and to receive ultrasonic waves reflected from the three-dimensional fields corresponding to the different moved positions.

The probe position sensing unit 12b senses the position of the ultrasonic probe 11. The probe position sensing unit 12b includes, for example, a magnetic sensor (not shown), a magnetic generator (not shown), and a probe position arithmetic unit (not shown). The magnetic sensor included in the probe position sensing unit 12b is disposed in the ultrasonic probe 11. The magnetic sensor and the magnetic generator each have three coils that are orthogonal to one another. The magnetic generator induces magnetic fields, and the magnetic sensor senses the magnetic fields induced by the magnetic generator. When the magnetic sensor senses the magnetic fields induced by the magnetic generator, the magnetic sensor generates induction fields that inversely correlate the slopes of the coils with respect to the directions of the magnetic fields, and transfers induced currents, whose values are proportional to the field strengths, to the probe position arithmetic unit. The probe position arithmetic unit calculates the position of the ultrasonic probe 11 and the slope thereof on the basis of the induced currents generated by the magnetic sensor. Thereafter, the probe position sensing unit 12b transfers data, which represents the sensed results, to the image construction unit 13.

According to the present embodiment, the probe position sensing unit 12b sequentially senses, for example, a first position on a subject to be scanned with the ultrasonic probe 11, and a second position to be scanned with the ultrasonic probe 11 manually moved by an operator. Thus, the probe position sensing unit 12b sequentially senses a plurality of positions to be scanned with the ultrasonic probe 11 manually moved by an operator. Thereafter, the probe position sensing unit 12b transfers positional data, which represents the positions of the ultrasonic probe 11 moved by the operator, to the image construction unit 13.

The image construction unit 13 constructs an image of a subject on the basis of an echo signal acquired by the transceiver 12a. The image construction unit 13 comprises, for example, a computer and a program. In response to a command issued from the control unit 301, the image construction unit 13 performs image processing on the basis of the echo signals sent from the transceiver 12a, and time-sequentially constructs images of subject's sections frame by frame. The image construction unit 13 is connected to the memory unit 14, and sequentially transfers the constructed frame images to the memory unit 14.

According to the present embodiment, the image construction unit 13 constructs a first image of a three-dimensional field corresponding to a first position on a subject on the basis of a first echo signal received by the transceiver 12a. Herein, the image construction unit 13 constructs the first image in real time with a scan that is performed in order to produce the first echo signal. Furthermore, the image construction unit 13 constructs a second image of a three-dimensional field corresponding to a second position on the basis of a second echo signal received by the transceiver 12a. Herein, the image construction unit 13 constructs the second image in real time with a scan that is performed in order to produce the second echo signal.

To be more specific, the image construction unit 13 constructs as the first and second images C-mode images that are images of sections separated from the contact surface of the ultrasonic probe 11, which is brought into contact with a subject, by substantially the same distance in a direction in which ultrasonic waves are propagated. In other words, images of transverse planes are constructed as the first and second images. Thus, the image construction unit 13 sequentially constructs C-mode images in real time on the basis of echo signals produced by scanning a plurality of positions with the ultrasonic probe 11 that is manually moved by an operator. The image construction unit 13 sequentially transfers the constructed C-mode images as the first and second images to the memory unit 14 so that the images will be stored in the memory unit. Moreover, the image construction unit 13 joins the first and second images constructed in the form of C-mode images so that the images will be associated with the first and second positions which are scanned, and thus constructs joint image. Herein, the image construction unit 13 aligns the first and second images according to data that represents the positions of the ultrasonic probe 11 sensed by the probe position sensing unit 12b, and joins the images to construct the joint image. In other words, the image construction unit 13 reads a plurality of C-mode images, which are sequentially constructed as mentioned above, from the memory unit 14, aligns the C-mode images, and joins them to construct the joint image. Thereafter, the image construction unit 13 transfers the constructed joint image to the memory unit 14 so that the joint image will be stored in the memory unit 14.

The memory unit 14 comprises, for example, a cine memory and a hard disk drive (HDD), and preserves images constructed by the image construction unit 13. The memory unit 14 is connected to the image construction unit 13. In response to a command issued from the control unit 301, a plurality of frame images constructed by the image construction unit 13 is temporarily stored in the cine memory, and then transferred to and stored in the HDD. For example, the memory unit 14 preserves frame images, which are equivalent to a motion picture that lasts for two min, in the cine memory. The frame images constituting the motion picture that lasts for two min are transferred to and stored in the HDD. Moreover, the cine memory included in the memory unit 14 is connected to the display unit 121. The frames constituting the motion picture and being stored in the cine memory are sequentially displayed on the display unit 121 in real time. The HDD included in the memory unit 14 is also connected to the display unit 121. In response to a command an operator enters at the operating unit 302, image data that represents frame images constituting a motion picture and being stored in the HDD is transmitted to the display unit 121. Consequently, the images are displayed on the display unit 121 according to the image data.

According to the present embodiment, in the memory unit 14, the first image which the image construction unit 13 has constructed in the form of a C-mode image on the basis of the first echo signal received by the transceiver 12a is stored in the cine memory. Thereafter, the second image which the image construction unit 13 has constructed in the form of a C-mode image on the basis of the second echo signal received by the transceiver 12a is stored in the cine memory. Thus, the memory unit 14 preserves a plurality of C-mode images, which the image construction unit 13 has sequentially constructed based on echo signals produced by scanning a plurality of positions with the ultrasonic probe 11 that is manually moved by an operator, sequentially in the cine memory in real time. Moreover, the memory unit 14 preserves a joint image that is constructed by joining the first and second images sequentially constructed as mentioned above by the image construction unit 13. Namely, the memory unit 14 sequentially updates and preserves the joint image constructed by joining a plurality of C-mode images sequentially constructed by the image construction unit 13. The image construction unit 13 transmits the constructed joint image to the display unit 121 so that the joint image will be displayed on a display surface.

The display unit 121 receives an image, which is constructed by the image construction unit 13, from the memory unit 14, and displays it. The display unit 121 comprises, for example, a graphic display and a digital scan converter (DSC). The display unit 121 is connected to the memory unit 14. In response to a command issued from the control unit 301, the DSC converts an image stored in the cine memory included in the memory unit 14 into a display signal, and the image constructed by the image construction unit 13 is displayed on the display screen of the graphic display in real time. Moreover, the display unit 121 is connected to the HDD included in the memory unit 14. In response to a command an operator enters at the operating unit 302, the display unit 121 receives image data representing frame images, which constitute a motion picture and are stored in the HDD, and displays the images on the screen thereof.

In the present embodiment, the display unit 121 displays a joint image, which is constructed by the image construction unit 13, on the display surface thereof in real time with a scan.

The control unit 301 comprises, for example, a computer and programs, and is connected to the other components. The control unit 301 applies control signals to the components according to an operating signal sent from the operating unit 302 so as to control the components.

The operating unit 302 comprises input devices, for example, a keyboard, a touch panel, a trackball, a footswitch, and an audio input device. An operator enters operational information at the operating unit 302, and the operating unit 302 transfers a command to the control unit 301 accordingly.

The diagnostic ultrasound apparatus 1 in accordance with the present embodiment is equivalent to a diagnostic ultrasound apparatus in accordance with the present invention. An ultrasonic probe 11 included in the present embodiment is equivalent to an ultrasonic probe included in the present invention. The transceiver 12a included in the present embodiment is equivalent to a transceiver included in the present invention. The position sensing unit 12b included in the present embodiment is equivalent to a position sensing unit included in the present invention. The image construction unit 13 included in the present embodiment is equivalent to an image construction unit included in the present invention. The display unit 121 included in the present embodiment is equivalent to a display unit included in the present invention.

Now, actions to be performed in the diagnostic ultrasound apparatus 1 in accordance with the present embodiment of the present invention will be described below.

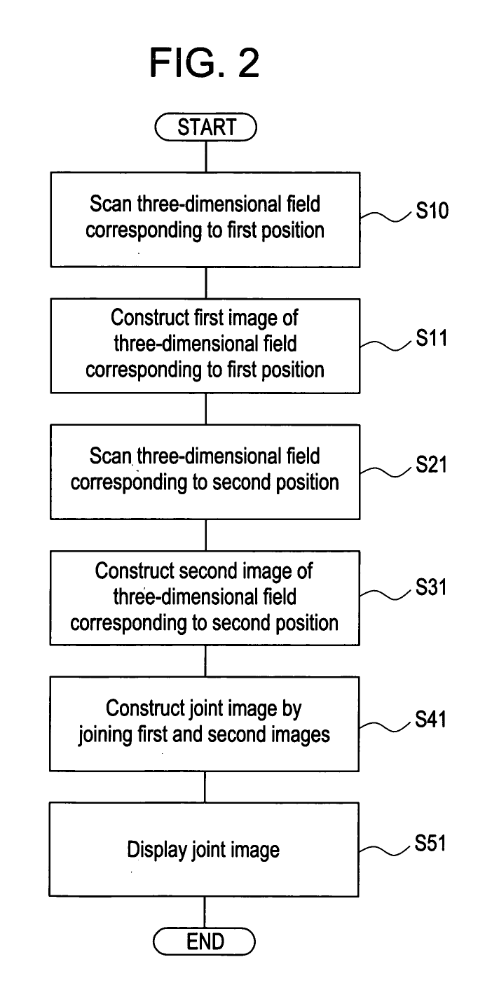

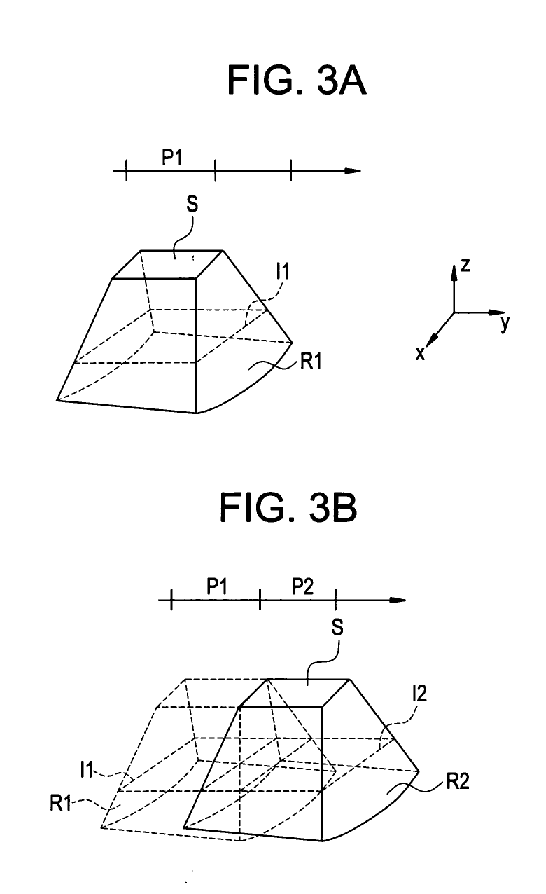

FIG. 2 and FIG. 3 are concerned with actions to be performed when the diagnostic ultrasound apparatus 1 scans a subject. FIG. 2 is a flowchart describing actions to be performed when the diagnostic ultrasound apparatus 1 in accordance with the present embodiment scans a subject and displays an image of the subject. FIG. 3 includes perspective views showing scans of a subject performed with the ultrasonic probe 11 included in the diagnostic ultrasound apparatus 1. FIG. 3a shows a scan of a three-dimensional field R1 corresponding to a first position P1 on a subject, and FIG. 3b shows a scan of a three-dimensional field R2 corresponding to a second position P2 on the subject.

As described in FIG. 2, first, the three-dimensional field R1 corresponding to the first position P1 on a subject is scanned (S11).

As shown in FIG. 3a, an operator brings the contact surface S of the ultrasonic probe 11 into contact with the first position P1 on a subject. The transceiver 12a acquires a first echo signal E1 produced by performing a scan to transmit ultrasonic waves from the ultrasonic probe 11 to the three-dimensional field R1 corresponding to the first position P1 on the subject and to receive the ultrasonic waves reflected from the three-dimensional field R1 corresponding to the first position P1. At this time, the probe position sensing unit 12b senses the first position P1 on the subject scanned with the ultrasonic probe 11.

Thereafter, as described in FIG. 2, a first image I1 of the three-dimensional field R1 corresponding to the first position P1 is constructed (S21).

Herein, as shown in FIG. 3a, the image construction unit 13 constructs as the first image I1 a C-mode image of the three-dimensional field R1 corresponding to the first position P1 on the subject according to the first echo signal E1 received by the transceiver 12a. Herein, the image construction unit 13 constructs the first image I1 in real time with the scan performed in order to produce the first echo signal E1. The image construction unit 13 then transfers the first image I1 to the memory unit 14 so that the first image will be stored in the memory unit.

Thereafter, as described in FIG. 2, the three-dimensional field R2 corresponding to the second position P2 on the subject is scanned (S31).

Herein, as shown in FIG. 3b, the operator slides the ultrasonic probe 11 in the direction y of the major axis of the contact surface S so as to move it from the position P1, and then brings the contact surface S of the ultrasonic probe 11 into contact with the position P2 on the subject. The transceiver 12a acquires a second echo signal E2 produced by performing a scan to transmit ultrasonic waves to the three-dimensional field R2 corresponding to the second position P2 on the subject and to receive the ultrasonic waves reflected from the three-dimensional field R corresponding to the second position P2. At this time, the probe position sensing unit 12b senses the position P2 on the subject scanned with the ultrasonic probe 11.

Thereafter, as described in FIG. 2, a second image I2 of the three-dimensional field R2 corresponding to the second position P2 is constructed (S41).

Herein, as shown in FIG. 3b, the image construction unit 13 constructs as the second image I2 a C-mode image of the three-dimensional field R2 corresponding to the second position P2 on the subject according to the second echo signal E2 received by the transceiver 12a. Herein, the image construction unit 13 constructs the second image I2 in real time with the scan performed in order to produce the second echo signal E2. The image construction unit 13 then transfers the second image I2 to the memory unit 14 so that the second image will be stored in the memory unit.

Thereafter, as described in FIG. 2, the first image I1 and second image I2 are joined to construct a joint image IK (S51).

Herein, the image construction unit 13 joins the first and second images I1 and I2 constructed in the form of C-mode images so that the first and second images will be associated with the first and second positions P1 and P2 on the subject, and thus constructs the joint image IK. In the present embodiment, the image construction unit 13 aligns the first and second images I1 and I2 according to data that represents the positions of the ultrasonic probe 11 sensed by the probe position sensing unit 12b, and joins the images to construct the joint image.

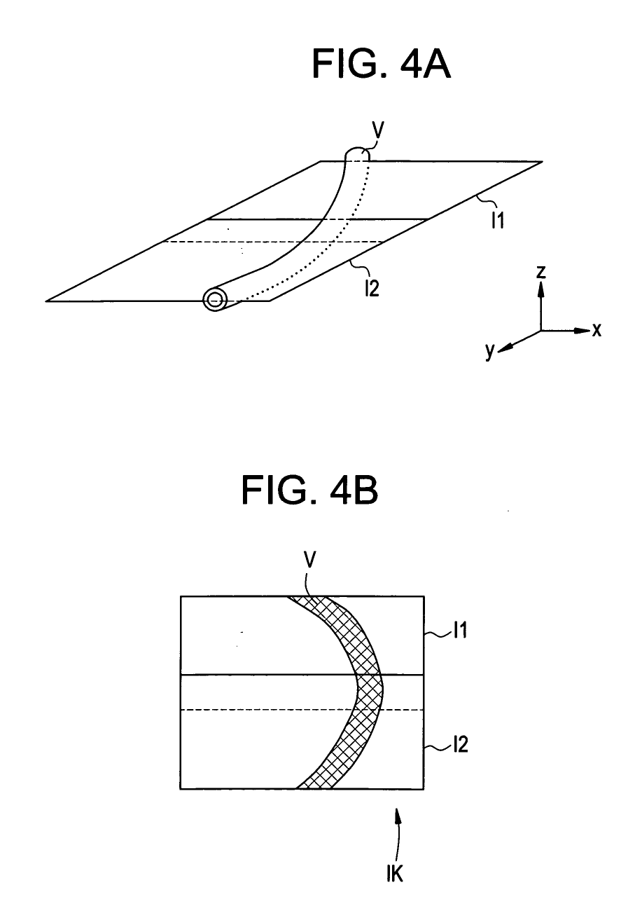

FIG. 4 shows construction of the joint image IK by joining the first and second images I1 and I2. Herein, FIG. 4a is a perspective view showing the relationship between a plane associated with the first and second images I1 and I2 constructed in the form of C-mode images and a subject's blood vessel visualized in the C-mode images. FIG. 4b shows the joint image IK constructed by joining the first and second images I1 and I2.

As shown in FIG. 4, the first and second images I1 and I2 are aligned so that the images will be associated with the respective positions on the subject, and then joined to overlap. Thus, the joint image I1 is constructed. For example, the vascular image portions V of the first and second images I1 and I2 are coupled to each other in order to construct the joint image IK. The image construction unit 13 transfers the resultant joint image Ik to the memory unit 14 so that the joint image will be stored in the memory unit.

Thereafter, as described in FIG. 2, the joint image IK is displayed (S61).

Herein, the display unit 121 fetches the joint image IK constructed by the image construction unit 13 from the memory unit 14, and displays it on the display screen thereof in real time with the scan.

As described so far, according to the present embodiment, first, the first image I1 of the three-dimensional field R1 corresponding to the first position P1 is constructed based on the first echo signal E1 produced by performing a scan to transmit ultrasonic waves from the ultrasonic probe 11 to the three-dimensional field R1 corresponding to the first position P1 on a subject and to receive the ultrasonic waves reflected from the three-dimensional field R1 corresponding to the first position P1. Herein, a C-mode image is constructed as the first image I1. Thereafter, the second image I2 of the three-dimensional field R2 corresponding to the second position P2 is constructed based on the second echo signal E2 produced by performing a scan to transmit ultrasonic waves to the three-dimensional field R corresponding to the second position P2 from the ultrasonic probe 11 that is moved from the first position P1 on the subject to the second position P2 and to receive the ultrasonic waves reflected from the three-dimensional field R2 corresponding to the second position P2. Herein, a C-mode image is constructed as the second image I2 in the same manner as the first image I1. Thereafter, as mentioned above, the first and second images I1 and I2 constructed in the form of C-mode images are joined so that the images will be associated with the first and second positions P1 and P2 respectively, whereby the joint image IK is constructed. The joint image IK is then displayed on the display surface of the display unit 121. The present embodiment constructs and displays the joint image IK that represents three-dimensional fields scanned by moving the ultrasonic probe 11. Consequently, the present embodiment makes it possible to observe the whole of a diagnostic region covering a wide range and to improve diagnostic efficiency.

### SECOND EMBODIMENT

The second embodiment of the present invention will be described below.

The present embodiment is identical to the first embodiment except the action of the image construction unit 13. An iterative description will be omitted.

According to the present embodiment, the image construction unit 13 constructs as first and second images three-dimensional images that represent a subject three-dimensionally.

To be more specific, the image construction unit 13 constructs as the first and second images three-dimensional images of voluminal fields in a subject juxtaposed in a direction in which ultrasonic waves are transmitted through the contact surface of the ultrasonic probe 11 that is brought into contact with the subject. Namely, so-called voluminal images are constructed as the first and second images. Herein, the image construction unit 13 sequentially constructs three-dimensional images in real time on the basis of echo signals produced by scanning a plurality of positions with the ultrasonic probe 11 that is manually moved by an operator. The image construction unit 13 sequentially transmits the three-dimensional images constructed as the first and second images to the memory unit 14 so that the images will be stored in the memory unit. Moreover, the image construction unit 13 joins the first and second images constructed in the form of the three-dimensional images so that the images will be associated with the first and second positions respectively that are scanned, whereby a joint image is constructed. Herein, similarly to the first embodiment, the image construction unit 13 aligns the first and second images according to data that represents the positions of the ultrasonic probe 11 sensed by the probe position sensing unit 12b, and joins the images to construct the joint image. The image construction unit 13 transfers the resultant joint image to the memory unit 14 so that the image will be stored in the memory unit.

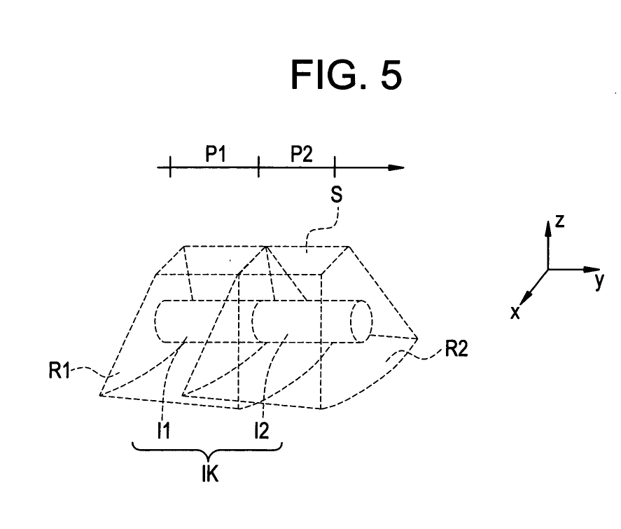

FIG. 5 is a perspective view showing the joint image constructed by the image construction unit 13 included in the present embodiment.

As shown in FIG. 5, first, the three-dimensional field R1 corresponding to the first position P1 is scanned in order to construct the first image I1 of the three-dimensional field R1 in the form of a three-dimensional image. Thereafter, the three-dimensional field R2 corresponding to the second position P2 on the subject is scanned in order to construct the second image I2 of the three-dimensional field R2 in the form of a three-dimensional image. Herein, for example, the first and second images I1 and I2 representing columnar fields in the subject are constructed in the form of three-dimensional images by performing rendering after producing three-dimensional data items on the basis of echo signals produced based on echoes returned from the respective fields. After the first and second images I1 and I2 of the columnar fields in the subject are constructed in the form of three-dimensional images, the first and second images I1 and I2 are joined to construct the joint image IK that appears three-dimensionally.

As mentioned above, in the present embodiment, similarly to the first embodiment, the first and second images I1 and I2 are constructed. Herein, the first and second images I1 and I2 are constructed in the form of three-dimensional images. Thereafter, the first and second images I1 and I2 are joined to construct the joint image IK. The joint image IK is then displayed on the display surface of the display unit 121. Consequently, the present embodiment constructs and displays the joint image IK representing three-dimensional fields scanned by moving the ultrasonic probe 11. Thus, the present embodiment makes it possible to observe the whole of a diagnostic region covering a wide range and to improve diagnostic efficiency.

Incidentally, the present invention is not limited to the aforesaid embodiments. Various variant modes can be adopted.

For example, in the embodiments, a probe capable of mechanically three-dimensionally scanning a subject can be adopted as the ultrasonic probe 11.

Moreover, for example, in the embodiments, when the first and second images I1 and I2 are joined to construct the joint image IK, the first and second images I1 and I2 may be aligned based on the correlational values between the pixels contained in the first image I1 and those contained in the second image I2. Thus, the joint image IK may be constructed.

Moreover, in the embodiments, the ultrasonic probe 11 is slid rectilinearly in the direction x of the major axis of the contact surface S in order to scan a subject. The present invention is not limited to this mode. The present invention can be applied to a case where, for example, the ultrasonic probe 11 is rectilinearly slid in the direction of the minor axis of the contact surface S in order to scan a subject. Moreover, the present invention can be applied to a case where, for example, the ultrasonic probe 11 is rotationally slid in order to scan a subject.

Many widely different embodiments of the invention may be configured without departing from the spirit and the scope of the present invention. It should be understood that the present invention is not limited to the specific embodiments described in the specification, except as defined in the appended claims.

1. An ultrasonic image construction method for constructing an image of a subject on the basis of an echo signal produced by transmitting ultrasonic waves from an ultrasonic probe to the subject and receiving the ultrasonic waves reflected from the subject using the ultrasonic probe, comprising:
a first step of constructing a first image of a three-dimensional field corresponding to a first position on a subject on the basis of a first echo signal produced by performing a scan to transmit ultrasonic waves from the ultrasonic probe to the three-dimensional field corresponding to the first position and to receive ultrasonic waves reflected from the three-dimensional field corresponding to the first position;
a second step of constructing a second image of a three-dimensional field corresponding to a second position on the basis of a second echo signal produced by performing a scan to transmit the ultrasonic waves to the three-dimensional field corresponding to the second position from the ultrasonic probe that is moved from the first position on the subject to the second position and to receive the ultrasonic waves reflected from the three-dimensional field corresponding to the second position, and then constructing a joint image by joining the first image constructed at the first step and the second image so that the first and second images will be associated with the first and second positions respectively.

2. The ultrasonic image construction method according to claim 1, wherein at the first and second steps, C-mode images are constructed as the first and second images.

3. The ultrasonic image construction method according to claim 1, wherein at the first and second steps, three-dimensional images are constructed as the first and second images.

4. The ultrasonic image construction method according to claim 1, further comprising:
a third step of sensing the first position scanned at the first step; and
a fourth step of sensing the second position scanned at the second step,
wherein at the second step, the first and second images are joined based on pieces of information on the first position sensed at the third step and the second position sensed at the fourth step in order to construct the joint image.

5. The ultrasonic image construction method according to claim 1, further comprising a fifth step of displaying the image constructed at the second step on a display surface.

6. A diagnostic ultrasound apparatus for constructing an image of a subject on the basis of an echo signal produced by transmitting ultrasonic waves from an ultrasonic probe to a subject and receiving the ultrasonic waves reflected from the subject using the ultrasonic probe, the diagnostic ultrasound apparatus comprising:
a transceiver that acquires a first echo signal produced by performing a scan to transmit the ultrasonic waves from the ultrasonic probe to a three-dimensional field corresponding to a first position on the subject and to receive the ultrasonic wave reflected from the three-dimensional field corresponding to the first position, and then acquires a second echo signal produced by performing a scan to transmit the ultrasonic waves to a three-dimensional field corresponding to a second position from the ultrasonic probe that is moved to the second position adjoining the first position on the subject and to receive the ultrasonic waves reflected from the three-dimensional field corresponding to the second position; and
an image construction device that constructs a first image of the three-dimensional field corresponding to the first position on the basis of the first echo signal, and constructs a second image of the three-dimensional field corresponding to the second position on the basis of the second echo signal,
wherein the image construction device joins the first and second images so that the first and second images will be associated with the first and second positions respectively.

7. The diagnostic ultrasound apparatus according to claim 6, wherein the image construction device constructs C-mode images as the first and second images.

8. The diagnostic ultrasound apparatus according to claim 6, wherein the image construction device constructs three-dimensional images as the first and second images.

9. The diagnostic ultrasound apparatus according to claim 6, further comprising a position sensing device that senses the position of the ultrasonic probe,
wherein the image construction device joins the first and second images according to the positions of the ultrasonic probe sensed by the position sensing device, and thus constructs the joint image.

10. The diagnostic ultrasound apparatus according to claim 6, further comprising a display device for displaying the joint image constructed by the image construction device.
